SEQUENCE LISTING (25 sequences)

SEQ ID NO: 1 - protein, 268 aa, synthetic construct
MKTTFAALIVSLTSIVSAYPITGDVVNCRTGPGTSYAIKTSYKKSHDISISCQTTGTSVNGNNIWDKTADGCYVADYYVKTGSSGFVTKKCSASGGGGSSSGSYCKTINGAGVDLIAKWEGFVASPKPDPIGLPTVGYGHLCQQKNCAEVKYKFPLTKTTAKELLLDDLPKYTKCLADVLNSKPKLNANQWAALSSWVFNVGCGNAKTSTLVKRLNNGEAPNTVAAQELPKWRMAGGKVMPGLEARRKDEVKLFKTASSKEAYPKCQA

SEQ ID NO: 2 - protein, 183 aa, synthetic construct
MSSSLKFLGVLLPALAVACTGPPVNQNGLNLIKSFESFQPSVYDDGFGNPTIGYGHLCGDATCSEVTYPKPLSEADASRLLADDLVSYQDALTNALADPVTLNDNQYAALVSWTFNIGNGNMQKSDLVARMNKGENVATVAHDELPQWNKANGQVVNGLTRRRKAELDLFDAPAIYGALPVPC

SEQ ID NO: 3 - protein, 185 aa, synthetic construct
MPSNTIKFILAALPALAAAACSGPDVNEATIKLMKGYESWEADVYDDGYGNPTVGYGHLCDDWSCSDVSYDIPLSESDGEKLFAEDIVAYQNGVVAALSDDVTLNDNQYGALVSWCFNVGTGAVAESTLAKRLNNGEDPDTVAEEELPKWVYANGAPSEGLKNRRAAELKLFTTSSDTEALPVDC

SEQ ID NO: 4 - protein, 184 aa, synthetic construct
MQRTVIAALAALASVASAQCNIPDVNTATINLITEFEGFRANIYTDPTGNPTVGIGHLCRDSRCSDVSYPIPLSQANGKKLLADDLGTYEECLTMMTNSRVVLNLNEFGALVSWAFNMGCGAAKGSTLIKRLNAGEKPQTVLPQELPKWVNGNGQVLPGLVRRRNAEVELARTATSSPALPACS

SEQ ID NO: 5 - protein, 228 aa, synthetic construct
MKFLTPLAVQLAALASMASATVPGFDISHYQATVDFAKAYADGARFVIIKATEGTTYTDPSFSDHYTKATNAGFIRGGYHFAQPASSSGAAQANYFLKHGGGWSADGITLPGMLDLEYAPSGDSCYGLSASAMVSWINDFVNTYHAATTQYPLIYTSTSWWQLCTGNNGSFGSKSPLVIARYASSVGALPNGWSVYTIWQNSDASPWGGDNDIFNGNLAQLQKIARGS

SEQ ID NO: 6 - protein, 173 aa, synthetic construct
MKFSIAAIALFTLSAVALPATVVRRGASAATVNLISEVEDFRADFYDMMGHKTIGYGHDCVAKKDCDSINAPISNAQGDEILHKDLVGFEQCVCALPNANALNANQYGALVSYAFNTGCGGLQQAWTAAMTSKNFDSICADFPHTNTLNGVLDNRRKKEAALCSTPTTQMSGC

SEQ ID NO: 7 - protein, 173 aa, synthetic construct
MKFSIAAIALFTLSAVALPATVVRRGASAATVSLIGEVEGFRADFYDMMGHKTIGYGHDCVAKQDCDSIKAPISNAQGDEILQKDLAGFEQCVCALPNAKALNANQYGALVSYAFNTGCGGLQQAWTAAMTSKNFDSICADLPHTNTLNGVLDNRRKKEAALCSTPTTQMCGC

SEQ ID NO: 8 - protein, 270 aa, synthetic construct
MKTTFAALIVSLTSIVSAYPITGDVVNCRTGPGTSYAIKTSYKKSHDISISCQTTGTSVNGNNIWDKTADGCYVADYYVKTGSSGFVTKKCSASGGGGGGSSSGSYCKTINGAGVDLIAKWEGFVASPKPDPIGLPTVGYGHLCQQKNCAEVKYKFPLTKTTAKELLLDDLPKYTKCLAEVLNSKPKLNANQWAALSSWVFNVGCGNAKTSTLVKRLNNGEAPNTVAAQELPKWRMAGGKVMPGLEARRKDEVKLFKTASSKEAYPKCQA

SEQ ID NO: 9 - protein, 267 aa, synthetic construct
MKTAFAALVFSLASIVSAYPITGDVVNCRTGPGTSYAIKKSYKKNQDISISCQTAGTSVNGNSIWDKTADGCYVADYYVKTGSSGYVTKKCSASSGGGSSSSSYCKTINTAGVDLIAKWEGFVASPKPDPIGLPTVGYGHLCQQKNCREVKYKFPLTKTTAKELLLDDLPKYTKCLADYLNDKPKLNANQWAALTSWVFNVGCGNAKTSTLVKRLNNGEAANTVAAEELPKWRMAGGKVLPGLEARRKDEVKLFKTASSKQAYPKCQ

SEQ ID NO: 10 - protein, 267 aa, synthetic construct
MKTAFAALVFSLASIVSAYPITGDVVNCRTGPGTSYAIKKSYKKNQDISISCQTAGTSVNGNSIWDKTADGCYVADYYVKTGSSGYVTKKCTASSGGGSSSSSYCKTINTAGVDLIAKWEGFVASPKPDPIGLPTVGYGHLCQQKNCREVKYKFPLTKTTAKELLLDDLPKYTKCLADYLNDKPKLNANQWAALTSWVFNVGCGNAKTSTLVKRLNNGEAANTVAAEELPKWRMAGGKVLPGLEARRKDEVKLFKTASSKQAYPKCQ

SEQ ID NO: 11 - protein, 182 aa, synthetic construct
MSSFKLLAALLPALAVACTGPPVNQNGLNLIKSFESFQPSVYDDGFGNPTIGYGHLCGDATCSEVTYPKPLSEADASALLAGDLVSYQDALTNALADPVTLNDNQYAALVSWTYNVGNGNMQKSDLVARMNKGEDVAAVAHDELPQWNKANGQVVNGLTRRRNAELGLFDAPAIYGALPVPC

SEQ ID NO: 12 - protein, 216 aa, synthetic construct
MKAISLLALPSLAYAAVQGFDISNWQPTVDYDGAYASGARFAMIKATEGTTYIDPLFNTHYPGATSAGLIRGGYHFAHPDSSSGSAQASYFLAHGGGWSADGLTLPGMLDLEAGCYGLSPSAMTSWIRDFGETYKDATGRYPMIYTTTSWWTECTANNADFGDYPLVLARWASSPGTLPASWDYYSFWQNSDQYAFGGDSQVWNGSEERLRVFASG

SEQ ID NO: 13 - DNA, 804 bp, synthetic construct
  source 1..804: mol_type = other DNA
atgaagacgacgtttgcggctcttattgtgtcattgacctccattgtctctgcataccccatcaccggcgacgtcgtcaactgccgcactggccctggcaccagctacgccatcaagaccagctacaagaagtcccacgacatcagcatcagctgccagaccaccggcaccagcgtcaacggcaacaacatctgggacaagaccgccgacggctgctacgtcgccgactactacgtcaagaccggcagcagcggcttcgtcaccaagaagtgcagcgcctctggcggcggcggcagctccagcggcagctactgcaagaccatcaacggcgctggcgtcgacctgatcgccaagtgggagggcttcgtcgctagccccaagcctgatcctatcggcctgcctaccgtcggctacggccacctctgccagcagaagaactgcgccgaggtcaagtacaagtttcccctgaccaagacgaccgccaaggagctgctcctcgacgacctgcctaagtacaccaagtgcctcgccgacgtcctcaacagcaagcccaagctcaacgccaaccagtgggccgctctcagcagctgggtctttaacgtcggctgcggcaacgccaagacgagcaccctcgtcaagcgcctcaacaacggcgaggcccctaacaccgtcgccgctcaggagctgcccaagtggcgcatggctggcggcaaggtcatgcccggccttgaggcccgacgcaaggacgaggtgaagctgttcaagacggccagctccaaggaggcttaccccaagtgccaggcc

SEQ ID NO: 14 - DNA, 549 bp, synthetic construct
  source 1..549: mol_type = other DNA
atgtcatccagcctcaagttcctgggggtcctgctgccggcgctcgccgtcgcttgcaccggccctcctgtcaaccagaacggcctcaacctcatcaagtccttcgagagcttccagcccagcgtctacgacgacggcttcggcaaccccaccatcggctacggccacctctgcggcgacgctacttgcagcgaggtcacctaccccaagcccctgtccgaggccgacgcttctcgcctcctggctgacgacctcgtctcctaccaggacgccctcaccaacgccctggccgatcctgtcaccctcaacgacaaccagtacgccgccctcgtcagctggaccttcaacatcggcaacggcaacatgcagaagagcgacctggtcgcccgcatgaacaagggcgagaacgtcgccaccgtcgcccacgatgagctgcctcagtggaacaaggccaacggccaggtcgtcaacggcctgacccgccgacgaaaggccgagctcgacctgttcgacgcccccgctatctacggcgccctgcctgtcccttgc

SEQ ID NO: 15 - DNA, 555 bp, synthetic construct
  source 1..555: mol_type = other DNA
atgccttcgaacaccattaagttcatcctcgctgcgttgcccgccctggcggcagcggcttgcagcggacccgacgtcaacgaggccaccatcaagctcatgaagggttatgagagctgggaggccgacgtttacgacgacggctacgggaacccgacagtgggatatggccacctctgcgatgactggtcctgttcggacgtatcttacgacattccgctctcggagtcagatggcgagaagctgtttgcggaagatatcgtggcataccaaaacggcgtcgtggctgctctaagcgacgatgtgacgctgaacgataatcagtacggcgccttagtcagttggtgcttcaacgtcggcacgggggccgttgcggaatctacacttgccaagaggctcaacaacggcgaagatccggacaccgtcgccgaggaggagctccccaagtgggtctacgccaatggtgccccatccgaaggcttgaagaatcggcgcgcagcagagctgaaactgtttactactagctccgacacggaggccctgcctgtcgactgc

SEQ ID NO: 16 - DNA, 552 bp, synthetic construct
  source 1..552: mol_type = other DNA
atgcagcgcaccgtcattgccgcccttgctgctctggcctccgtggcgtctgctcagtgcaacatcccggatgtgaacactgcaacgatcaacctgatcaccgagtttgagggcttccgtgctaatatctacacggatccgacgggcaacccgacagtcggcattggccacctctgccgcgacagccggtgctctgacgtctcgtatcccatacctctctcccaagccaacggcaagaagctgcttgccgacgacctgggcacatacgaggaatgcctgaccatgatgaccaacagccgagttgttctgaatctcaacgagtttggggccctcgtgagctgggcgttcaacatgggctgcggtgcggccaaaggctccacgctcatcaagcggttgaatgcaggagagaagccccagacggtgctccctcaagaactacccaagtgggtcaatgggaacggacaggtcctgccaggtctcgtaagaaggcgcaacgcggaagtcgagttggcccgaaccgcgacttcaagtccagcactgcccgcctgttcg

SEQ ID NO: 17 - DNA, 684 bp, synthetic construct
  source 1..684: mol_type = other DNA
atgaagttcttgactccgcttgctgtgcagctcgccgccctggcgtccatggcctcggcgacagttcccggcttcgacattagccattaccaggcaaccgtcgacttcgccaaggcgtacgccgacggcgcccgcttcgtcatcatcaaggccacagaaggcaccacctacaccgaccccagctttagtgaccactatacgaaagctaccaacgccggtttcatccgaggaggctaccactttgcccagcccgcatcttctagcggggctgcacaggccaactacttcctaaagcacggcggtggctggtccgccgatggcataaccctgccgggcatgctcgacctggagtatgcaccttctggagattcatgctacggcctcagtgcatcagccatggtctcctggatcaacgactttgtcaacacgtaccatgccgcgacgacgcagtaccctctcatctacacatcgacgagctggtggcaattgtgcactggcaacaacgggtcctttggcagcaagagcccacttgttattgccaggtatgcttcgagcgtgggagctctgcccaatggatggtcggtatataccatctggcagaactcggatgcgtccccgtggggcggcgacaacgatatcttcaatgggaatctcgctcaactgcagaagattgcgcggggttca

SEQ ID NO: 18 - DNA, 519 bp, synthetic construct
  source 1..519: mol_type = other DNA
atgaagttctcgatagctgccattgccttgttcaccctgagcgccgtggcgctgccggcaactgttgttcggaggggcgcctctgccgccaccgtcaacctcatcagcgaggtcgaggacttccgcgccgacttctacgacatgatgggccacaagaccatcggctacggccacgactgcgtcgccaagaaggactgcgacagcatcaacgcccctatcagcaacgcccagggcgacgagatcctccacaaggacctcgtcggcttcgagcagtgcgtctgcgccctgcctaacgccaacgctctcaacgccaaccagtacggcgccctcgtcagctacgccttcaacaccggctgcggcggcctccagcaggcctggaccgccgccatgaccagcaagaacttcgactccatctgcgccgattttccccacaccaacacgctcaacggcgtcctcgacaaccgccgcaagaaggaggccgctctctgcagcacgcccaccacgcagatgagcggctgc

SEQ ID NO: 19 - DNA, 519 bp, synthetic construct
  source 1..519: mol_type = other DNA
atgaagttcagcatagctgccatcgccctcttcaccctctcagccgtcgccttgcctgccaccgtggttcgacggggcgcctctgccgccaccgtcagcctcatcggcgaggtcgagggcttccgcgccgacttctacgacatgatgggccacaagaccatcggctacggccacgactgcgtcgccaagcaggactgcgacagcatcaaggcccctatcagcaacgcccagggcgacgagatcctccagaaggacctggccggcttcgagcagtgcgtctgcgccctgcctaacgccaaggctctcaacgccaaccagtacggcgccctcgtcagctacgccttcaacaccggctgcggcggcctccagcaggcctggaccgccgccatgaccagcaagaacttcgactccatctgcgccgacctgcctcacaccaacacgctcaacggcgtcctcgacaaccgccgcaagaaggaggccgctctctgcagcacgcccaccacgcagatgtgcggctgc

SEQ ID NO: 20 - DNA, 810 bp, synthetic construct
  source 1..810: mol_type = other DNA
atgaagactacctttgccgctcttattgtttcgttgacgtccatcgtgtcagcatatcccatcacgggagatgtcgtcaactgccgcacgggacccggcacatcgtacgccattaagaccagctacaagaagagccacgacatcagcatatcctgccaaaccacggggaccagcgtcaatggcaacaacatctgggacaaaactgctgatggctgctacgtggccgactactacgtcaagacggggtcctcgggcttcgtcaccaagaagtgctcggcgtctggcggcgggggtggtggatcctcttccgggagctactgcaagacaatcaacggcgccggagtggatctcatcgcgaagtgggagggctttgttgcctcacctaagcccgacccgattggcttgccgacagttggctacggccatctatgccagcagaagaactgtgctgaggtaaagtacaagttccctctgaccaaaacgaccgccaaggaactgctgctcgacgacctgccgaagtatacgaaatgcctggccgaggtgttgaatagcaaaccaaagctgaacgcaaaccagtgggctgccctgtcgagctgggtcttcaacgtgggatgcggcaatgcgaagacctctacacttgtcaagcgactcaacaatggtgaggctcccaacaccgtggcagcgcaggagctcccaaaatggcgcatggccggcggcaaggtcatgcccggcctcgaggcgaggcggaaggacgaagtcaagctcttcaagacggccagttcaaaggaagcatatccgaaatgtcaagcc

SEQ ID NO: 21 - DNA, 801 bp, synthetic construct
  source 1..801: mol_type = other DNA
atgaagacggcctttgctgctcttgtttttagcctggcaagcattgtctccgcctaccccatcacaggcgatgtcgtgaattgtcgaactggtccgggaacctcctacgctatcaagaaaagctacaagaagaaccaagatattagcatctcgtgccagaccgcagggacgtccgtcaacggcaactccatctgggacaagactgcggacggctgctacgtggccgactactacgtcaagaccggtagttctggatatgtcaccaagaagtgcagcgcctcgtccggaggtggctcatctagttcatcgtactgcaagaccatcaacactgctggagtcgacttgatagccaaatgggagggcttcgtagccagccccaagcccgatcctattgggctgccaacagtgggctatgggcacttgtgccagcagaagaactgcagagaagtgaaatacaagttcccgctgactaaaacgacggcgaaggagctgctgctcgacgaccttccaaagtacacgaagtgcctcgccgattatctaaacgacaagccgaagctgaatgccaaccagtgggcggcgctcacatcctgggtgttcaacgtcggctgtggcaatgccaaaacctctaccctcgtcaagcggctcaacaacggcgaggcggcaaacacggttgccgccgaggagctccccaagtggcgcatggccggcggcaaggtcctgcctggcctggaagcgaggcgcaaggacgaggttaagctcttcaagacggcatcgtcgaagcaggcttatcccaagtgccaa

SEQ ID NO: 22 - DNA, 801 bp, synthetic construct
  source 1..801: mol_type = other DNA
atgaagacggcctttgccgcgctggtgttttcccttgcctcgattgtctcggcgtatcccatcaccggcgacgtcgtcaactgcaggacggggccggggacaagctatgccatcaagaaatcgtacaagaagaaccaagacatatccatcagctgccagacggctggcacgtctgtcaatggcaacagcatctgggacaagactgcggatggctgctacgtcgctgactactatgtgaaaactggtagtagtggatacgtgacgaagaagtgcaccgcatcttcaggtggaggaagctcgtcatcttcctactgcaagaccatcaacacggcgggagtcgacctcattgccaagtgggagggcttcgtggccagcccgaagcccgatcctattgggctccccacggttggctacggccacctctgccagcagaagaattgccgcgaagttaagtacaagttccctctgaccaagaccacagcaaaggagctcctcctggacgatttgccgaaatacaccaaatgcttggccgattatctcaacgacaagccaaagctgaacgccaaccagtgggctgctcttactagctgggtcttcaacgttggctgtggcaatgccaagacgtcgaccctcgtcaagcgacttaacaacggcgaggcagcaaacaccgtggcggccgaggagctgccaaaatggcgcatggccggcggcaaggtcctgcccggtctggaagccagacggaaggacgaggtaaagctcttcaagacagcttcctccaagcaggcgtaccctaagtgtcaa

SEQ ID NO: 23 - DNA, 546 bp, synthetic construct
  source 1..546: mol_type = other DNA
atgagctcattcaagctgcttgctgcattgctcccagcactggctgtggcctgcactggacctcccgtcaaccaaaacggtctgaacctcatcaagtcgtttgagtccttccagccgtctgtatacgacgacggcttcggcaatcccaccattggctatggccatctttgcggcgacgccacctgctccgaagtgacgtatcccaagcccttgtcggaagcggatgcatccgcactactggccggggatctggtcagctaccaggatgctctcaccaacgcgctcgccgaccccgtcacgctgaacgacaaccagtacgccgcgcttgtgagttggacgtacaacgtgggtaacggaaatatgcagaagagcgacctcgtcgcgcgcatgaacaagggcgaggacgtcgccgccgttgcgcacgacgagctccctcaatggaacaaagctaacggccaggtcgtcaatgggctcacaaggcgacggaatgccgagctgggactgtttgatgctccagccatctacggcgccttgccggttccgtgt

SEQ ID NO: 24 - DNA, 648 bp, synthetic construct
  source 1..648: mol_type = other DNA
atgaaagccatctccctccttgcactaccatctctcgcttatgctgctgtccagggcttcgacatttccaattggcaaccgacagtcgactacgacggcgcgtatgcgtccggtgcccgatttgccatgatcaaggcgaccgaaggcaccacctacatcgaccccctcttcaacacgcactaccctggcgctaccagcgccggcctcatacggggaggttaccacttcgcccatcccgattcttcgtcggggtcagcacaggcctcctacttcttggcgcacggcggaggctggtccgcagatgggctgacgctgcccggcatgctcgacctggaagccggctgctatggcctgagcccgtcggccatgacgagctggatccgcgacttcggagagacgtacaaggacgccaccgggagatatcccatgatttacacgactacatcatggtggaccgagtgcaccgccaacaacgccgattttggcgactatccactcgtgttggcaaggtgggcctcttccccgggcacactgcctgcgtcgtgggattactacagcttctggcagaatagcgaccagtacgcctttggtggagatagccaagtctggaacggcagcgaggagcgtcttcgcgtttttgcttcgggg

SEQ ID NO: 25 - DNA, 419 bp, synthetic construct
  source 1..419: mol_type = other DNA
ttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatctagataacttcgtataatgtatgctatacgaagttatactagagaagttcctatactttttagagaataggaacttcggaataggaacttcactagagatatcaagcaactacgtaaaactccatgagattgcagatgcggcccactggaatacaacatcctccgcaagtccga

(19) United States
(12) Patent Application Publication
(10) Pub. No.: US 2026/0159552 A1
(43) Pub. Date: Jun. 11, 2026

(54) BACTERICIDAL PROTEIN AND ENCODING GENE AND APPLICATION THEREOF

(71) Applicants: Wei WANG, Shanghai City (CN); Ningxia Sunson Industrial Group Co., Ltd., Yinchuan (CN)

(72) Inventors: Wei WANG, Shanghai City (CN); Tao SHEN, Yinchuan (CN)

(21) Appl. No.: 19/536,624

(22) Filed: Feb. 11, 2026

Related U.S. Application Data
(63) Continuation of application No. PCT/CN2025/102717, filed on Jun. 23, 2025.

(30) Foreign Application Priority Data
Jul. 24, 2024 (CN) 202410994914.8

(51) Int. Cl. C07K 14/00

(52) U.S. Cl. CPC C07K 14/001

(57) ABSTRACT

A bactericidal protein and an encoding gene and an application thereof are provided. An amino acid sequence of the bactericidal protein is any one of SEQ ID NO. 1 to SEQ ID NO. 12, or a sequence having identical function after 1 to 30 amino acid substitutions and/or deletions and/or additions to any one of SEQ ID NO. 1 to SEQ ID NO. 12, or a sequence having identical function after adding 1 to 20 amino acids to the C-terminus and/or N-terminus of any one of SEQ ID NO. 1 to SEQ ID NO. 12.

### CROSS-REFERENCE TO RELATED APPLICATIONS

This application is a continuation of International Patent Application No. PCT/CN2025/102717, filed on Jun. 23, 2025, and claims priority of Chinese Patent Application No. 202410994914.8, filed on Jul. 24, 2024. The contents of International Patent Application No. PCT/CN2025/102717 and Chinese Patent Application No. 202410994914.8 are hereby incorporated by reference.

### INCORPORATION BY REFERENCE STATEMENT

This statement, made under Rules 77 (b) (5) (ii) and any other applicable rule incorporates into the present specification of an XML file for a “Sequence Listing XML” (see Rule 831 (a)), submitted via the USPTO patent electronic filing system or on one or more read-only optical discs (see Rule 1.52 (e) (8)), identifying the names of each file, the date of creation of each file, and the size of each file in bytes as follows:
File name: SequenceListing.xml
Creation date: Feb. 9, 2026
Byte size: 33,284

### TECHNICAL FIELD

The present disclosure relates to the technical field of genetic engineering, and in particular to a bactericidal protein, its encoding gene, and application thereof.

### BACKGROUND

Microbial cell walls are composed of polysaccharides, primarily including peptidoglycan, glucan, mannan, chitin, and cellulose. Fungal genomes contain abundant unknown proteins with glycoside hydrolase activity. If they may hydrolyze the aforementioned polysaccharides, they may hydrolyze microbial cell walls, achieving a bactericidal effect. Known academic research has not reported relevant studies on fungal bactericidal proteins.

Trichoderma reesei is an important industrial production strain, complying with the Generally Regarded as Safe (GRAS) standard, and its fermentation-produced enzyme preparations have been widely used in industries such as food and feed. Known academic research has not reported that Trichoderma reesei strains may ferment and produce fungal bactericidal proteins.

The present disclosure intends to utilize Trichoderma reesei to secrete and express a bactericidal protein, thereby developing new technical ideas and providing new technical schemes for the field of pathogen eradication.

### SUMMARY

An object of the present disclosure is to provide a bactericidal protein, its encoding gene, and application thereof, to solve the problems existing in the aforementioned prior art. The bactericidal protein produced by the present disclosure may hydrolyze bacterial cell walls, causing lysis of Micrococcus luteus. Verification shows that the bactericidal protein may significantly inhibit or kill Gram-positive bacteria, Gram-negative bacteria, and yeasts, providing a new bactericidal protein preparation and a preparation method thereof for industries including daily chemicals, food, and feed.

To achieve the aforementioned object, the present disclosure provides the following scheme.

The present disclosure provides a bactericidal protein, where an amino acid sequence of the bactericidal protein is any one of SEQ ID NO. 1 to SEQ ID NO. 12, or a sequence having identical function after substitutions and/or deletions and/or additions of 1 to 30 amino acids to any one of SEQ ID NO. 1 to SEQ ID NO. 12, or a sequence having identical function after adding 1 to 20 amino acids to the C-terminus and/or N-terminus of any one of SEQ ID NO. 1 to SEQ ID NO. 12.

In the art, substitution with amino acids having similar or comparable properties typically does not alter the function of a protein. Adding one or multiple amino acids to the C-terminus and/or N-terminus also usually does not change the function of a protein. Therefore, in the present disclosure, the amino acid sequence of the bactericidal protein also includes variant forms of any one of SEQ ID NO. 1 to SEQ ID NO. 12 that have the same function as the bactericidal protein. These variant forms include: substitution and/or deletion and/or addition of multiple (1 to 30, 1 to 20, more optionally 1 to 10, even more optionally 1 to 5) amino acids, and addition of one or multiple (1 to 20, 1 to 10, more optionally 1 to 5) amino acids to the C-terminus and/or N-terminus of any one of SEQ ID NO. 1 to SEQ ID NO. 12.

The present disclosure also provides a gene encoding the aforementioned bactericidal protein, where a nucleotide sequence of the gene is any one of SEQ ID NO. 13 to SEQ ID NO. 24, or a sequence having more than 85% homology to any one of SEQ ID NO. 13 to SEQ ID NO. 24.

Due to the existence of codon degeneracy (degeneracy refers to a sequence produced after one or more codons are replaced by degenerate codons encoding the same amino acid), therefore, in the present disclosure, the nucleotide sequence of the gene encoding the bactericidal protein also includes a sequence having more than 85% homology to any one of SEQ ID NO. 13 to SEQ ID NO. 24.

The present disclosure also provides a recombinant expression vector, where the recombinant expression vector includes a nucleotide sequence encoding the aforementioned bactericidal protein, or includes the aforementioned gene.

The present disclosure also provides a recombinant microorganism, where the recombinant microorganism includes the aforementioned recombinant expression vector.

Optionally, a chassis microorganism of the recombinant microorganism is a Trichoderma species.

Optionally, the Trichoderma species is Trichoderma reesei QM6a, QM9414, RUT-C30, RL-P37, NG14, or PC-3-7.

The present disclosure also provides an application of the aforementioned gene, the aforementioned recombinant expression vector, or the aforementioned recombinant microorganism in producing a bactericidal protein.

The present disclosure also provides a method for producing a bactericidal protein, including fermenting the aforementioned recombinant microorganism, collecting a fermentation broth, and obtaining the bactericidal protein.

The present disclosure also provides an application of the bactericidal protein prepared by the aforementioned production method in preparing a bactericidal agent.

The present disclosure has the following beneficial effects.

The present disclosure discloses an amino acid sequence of a bactericidal protein, and a gene sequence encoding the bactericidal protein. Using the Trichoderma expression vector CBHIT1.0, the encoding gene of the bactericidal protein is transformed into the Trichoderma genome, successfully constructing a recombinant Trichoderma strain that secretes and expresses the bactericidal protein. This recombinant Trichoderma strain may be used for the production of the bactericidal protein. Experimental verification shows that the bactericidal protein produced by the present disclosure may significantly kill Gram-positive bacteria, Gram-negative bacteria, and yeasts, and its effect is superior to that of common bactericidal proteins in the prior art. The present disclosure provides a new bactericidal protein preparation and a preparation method thereof, and its application in pathogen eradication for industries including daily chemicals, food, and feed.

### BRIEF DESCRIPTION OF THE DRAWINGS

To illustrate the technical schemes in the embodiments of the present disclosure or in the prior art more clearly, the drawings required for use in the embodiments will be briefly described below. Apparently, the drawings in the following description are only some embodiments of the present disclosure. For those of ordinary skill in the art, other drawings may also be obtained from these drawings without creative effort.

FIG. 1 is a schematic diagram of the recombinant expression vector structure in Embodiment 1.

FIG. 2 is a schematic diagram of the construction process of the recombinant Trichoderma strain in Embodiment 2.

FIG. 3A shows the experimental results of the inhibition zone for bactericidal protein F5 and commercial lysozyme against Candida albicans in Embodiment 4.

FIG. 3B shows the experimental results of the inhibition zone for bactericidal protein F5 and commercial lysozyme against Staphylococcus aureus in Embodiment 4.

FIG. 3C shows the experimental results of the inhibition zone for bactericidal protein F5 and commercial lysozyme against Escherichia coli in Embodiment 4.

### DETAILED DESCRIPTION OF THE EMBODIMENTS

Various exemplary embodiments of the present disclosure are now described in detail, which detailed description should not be considered as limiting the disclosure, but rather as providing a more detailed description of certain aspects, characteristics, and embodiments of the present disclosure.

It should be understood that the terms described in the present disclosure are only for describing specific embodiments and are not intended to limit the disclosure. In addition, for numerical ranges in the present disclosure, it should be understood that each intermediate value between the upper limit and the lower limit of the range is also specifically disclosed. Every smaller range between any stated value or intermediate value within a stated range, and any other stated value or intermediate value within the range, is also included in the present disclosure. The upper and lower limits of these smaller ranges may be independently included or excluded from the range.

Unless otherwise stated, all technical and scientific terms used herein have the same meanings as commonly understood by one of ordinary skill in the art to which this disclosure pertains. Although only optional methods and materials are described in the present disclosure, any methods and materials similar or equivalent to those described herein may also be used in the practice or testing of the present disclosure. All documents mentioned in this specification are incorporated by reference to disclose and describe the methods and/or materials related to the documents. In case of any conflict with any incorporated document, the content of this specification shall prevail.

Various modifications and changes may be made to the specific embodiments of the description of the present disclosure without departing from the scope or spirit of the disclosure, which will be apparent to those skilled in the art. Other embodiments obtained from the description of the present disclosure will be apparent to those skilled in the art. The description and embodiments of the present disclosure are illustrative only.

Regarding the terms “comprise”, “include”, “have”, “contain”, and the like as used herein, they are all open-ended terms, meaning inclusion but not limited to.

In the embodiments of the method of the present disclosure, the formulations of the culture media and reagents are as follows.
(1) The formulation of Luria Bertani (LB) medium is: 5 grams of yeast extract, 10 grams of peptone, 10 grams of sodium chloride, made up to 1 liter with tap water, at natural pH.
(2) The formulation of potato dextrose agar (PDA) medium is: 200 grams of potato, 20 grams of glucose (or xylose), 20 grams of agar, made up to 1 liter with tap water, at natural pH.
(3) The formulation of the Trichoderma fermentation medium (1 liter) is: 30 grams of glucose, 10 grams of lactose, 10 grams of cellulose powder, 5 grams of wheat bran, 5 grams of corn steep liquor, 3 grams of peptone, 2 grams of yeast extract, 2.0 grams of KH₂PO₄, 0.34 grams of CaCl₂, 0.3 grams of MgSO₄·7H₂O, 1 milliliter of Mandels trace element solution, and 1 milliliter of Tween 80.
(4) The formulation of the Mandels trace element solution (1000×) is: 5 grams of FeSO₄·7H₂O, 2 grams of CoCl₂·6H₂O, 1.4 grams of ZnSO₄·7H₂O, 1.6 grams of MnSO₄·H₂O, made up to 1 liter with purified water.
(5) The protocol for Agrobacterium Tumefaciens-mediated conjugation transfer in Trichoderma is: the expression vector is electroporated into Agrobacterium tumefaciens, and then the successfully electroporated Agrobacterium tumefaciens is co-cultured with Trichoderma strains QM6a (ATCC 13631), QM9414 (ATCC 26921), RUT-C30 (ATCC 56765), RL-P37 (NRRL 15709), NG14 (ATCC 56767), and PC-3-7 (ATCC 66589) on induction medium (IM) plates (Covert et al. Agrobacterium Tumefaciens-mediated Transformation of Fusarium Circinatum. Mycol. Res. 105 (3): 259-264) for Agrobacterium tumefaciens-mediated conjugation transfer. After two days of co-culture, the mixture is transferred to PDA plates containing cefotaxime (300 micrograms per milliliter) and hygromycin B (75 micrograms per milliliter) for screening until mycelia and spores grow. Then polymerase chain reaction (PCR) verification is performed to prove that the grown colonies are correct transformants.

The plasmid extraction kit is purchased from AXYGEN, the gel recovery kit is purchased from MAGEN, the seamless cloning kit is selected from TransGen Biotech, and DNA restriction endonucleases and ligases are selected from NEB. Similar products from other companies may also be used as substitutes.

For the experimental methods in the following embodiments where specific conditions are not stated, they are generally carried out under conventional conditions, such as the conditions described in Molecular Cloning: A Laboratory Manual (New York: Cold Spring Harbor Laboratory Press, 1989).

The amino acid sequences of the bactericidal proteins in the following embodiments are as shown in SEQ ID NO. 1 to SEQ ID NO. 12.

SEQ ID NO. 1:
MKTTFAALIVSLTSIVSAYPITGDVVNCRTGPGTSYAIKTSYKKSHDISISCQTT
GTSVNGNNIWDKTADGCYVADYYVKTGSSGFVTKKCSASGGGGSSSGSYCKTINGAGV
DLIAKWEGFVASPKPDPIGLPTVGYGHLCQQKNCAEVKYKFPLTKTTAKELLLDDLPKYTKC
LADVLNSKPKLNANQWAALSSWVFNVGCGNAKTSTLVKRLNNGEAPNTVAAQEL
PKWRMAGGKVMPGLEARRKDEVKLFKTASSKEAYPKCQA.
SEQ ID NO. 2:
MSSSLKFLGVLLPALAVACTGPPVNQNGLNLIKSFESFQPSVYDDGFGNPTIG
YGHLCGDATCSEVTYPKPLSEADASRLLADDLVSYQDALTNALADPVTLNDNQYAALVSWTF
NIGNGNMQKSDLVARMNKGENVATVAHDELPQWNKANGQVVNGLTRRRKAEL
DLFDAPAIYGALPVPC.
SEQ ID NO. 3:
MPSNTIKFILAALPALAAAACSGPDVNEATIKLMKGYESWEADVYDDGYGN
PTVGYGHLCDDWSCSDVSYDIPLSESDGEKLFAEDIVAYQNGVVAALSDDVTLNDNQY
GALVSWCFNVGTGAVAESTLAKRLNNGEDPDTVAEEELPKWVYANGAPSEGLKNRRA
AELKLFTTSSDTEALPVDC.
SEQ ID NO. 4:
MQRTVIAALAALASVASAQCNIPDVNTATINLITEFEGFRANIYTDPTGNPTV
GIGHLCRDSRCSDVSYPIPLSQANGKKLLADDLGTYEECLTMMTNSRVVLNLNEFGALV
SWAFNMGCGAAKGSTLIKRLNAGEKPQTVLPQELPKWVNGNGQVLPGLVRRRNAEVE
LARTATSSPALPACS.
SEQ ID NO. 5:
MKFLTPLAVQLAALASMASATVPGFDISHYQATVDFAKAYADGARFVIIKAT
EGTTYTDPSFSDHYTKATNAGFIRGGYHFAQPASSSGAAQANYFLKHGGGWSADGITLP
GMLDLEYAPSGDSCYGLSASAMVSWINDFVNTYHAATTQYPLIYTSTSWWQLCTGNN
GSFGSKSPLVIARYASSVGALPNGWSVYTIWQNSDASPWGGDNDIFNGNLAQLQKIARGS.
SEQ ID NO. 6:
MKFSIAAIALFTLSAVALPATVVRRGASAATVNLISEVEDFRADFYDMMGHK
TIGYGHDCVAKKDCDSINAPISNAQGDEILHKDLVGFEQCVCALPNANALNANQYGAL
VSYAFNTGCGGLQQAWTAAMTSKNFDSICADFPHTNTLNGVLDNRRKKEAALCSTPTT
QMSGC.
SEQ ID NO. 7:
MKFSIAAIALFTLSAVALPATVVRRGASAATVSLIGEVEGFRADFYDMMGHK
TIGYGHDCVAKQDCDSIKAPISNAQGDEILQKDLAGFEQCVCALPNAKALNANQYGAL
VSYAFNTGCGGLQQAWTAAMTSKNFDSICADLPHTNTLNGVLDNRRKKEAALCSTPTT
QMCGC.
SEQ ID NO. 8:
MKTTFAALIVSLTSIVSAYPITGDVVNCRTGPGTSYAIKTSYKKSHDISISCQTT
GTSVNGNNIWDKTADGCYVADYYVKTGSSGFVTKKCSASGGGGGGSSSGSYCKTINGA
GVDLIAKWEGFVASPKPDPIGLPTVGYGHLCQQKNCAEVKYKFPLTKTTAKELLLDDLP
KYTKCLAEVLNSKPKLNANQWAALSSWVFNVGCGNAKTSTLVKRLNNGEAPNTVAAQ
ELPKWRMAGGKVMPGLEARRKDEVKLFKTASSKEAYPKCQA.
SEQ ID NO. 9:
MKTAFAALVESLASIVSAYPITGDVVNCRTGPGTSYAIKKSYKKNQDISISCQT
AGTSVNGNSIWDKTADGCYVADYYVKTGSSGYVTKKCSASSGGGSSSSSYCKTINTAG
VDLIAKWEGFVASPKPDPIGLPTVGYGHLCQQKNCREVKYKFPLTKTTAKELLLDDLPK
YTKCLADYLNDKPKLNANQWAALTSWVFNVGCGNAKTSTLVKRLNNGEAANTVAAE
ELPKWRMAGGKVLPGLEARRKDEVKLFKTASSKQAYPKCQ.
SEQ ID NO. 10:
MKTAFAALVESLASIVSAYPITGDVVNCRTGPGTSYAIKKSYKKNQDISISCQT
AGTSVNGNSIWDKTADGCYVADYYVKTGSSGYVTKKCTASSGGGSSSSSYCKTINTAG
VDLIAKWEGFVASPKPDPIGLPTVGYGHLCQQKNCREVKYKFPLTKTTAKELLLDDLPK
YTKCLADYLNDKPKLNANQWAALTSWVFNVGCGNAKTSTLVKRLNNGEAANTVAAE
ELPKWRMAGGKVLPGLEARRKDEVKLFKTASSKQAYPKCQ.
SEQ ID NO. 11:
MSSFKLLAALLPALAVACTGPPVNQNGLNLIKSFESFQPSVYDDGFGNPTIGY
GHLCGDATCSEVTYPKPLSEADASALLAGDLVSYQDALTNALADPVTLNDNQYAALVS
WTYNVGNGNMQKSDLVARMNKGEDVAAVAHDELPQWNKANGQVVNGLTRRRNAEL
GLFDAPAIYGALPVPC.
SEQ ID NO. 12:
MKAISLLALPSLAYAAVQGFDISNWQPTVDYDGAYASGARFAMIKATEGTT
YIDPLFNTHYPGATSAGLIRGGYHFAHPDSSSGSAQASYFLAHGGGWSADGLTLPGMLD
LEAGCYGLSPSAMTSWIRDFGETYKDATGRYPMIYTTTSWWTECTANNADFGDYPLVL
ARWASSPGTLPASWDYYSFWQNSDQYAFGGDSQVWNGSEERLRVFASG.

The synthesis method of the bactericidal protein encoding gene provided in the embodiments of the present disclosure is as follows.

Amino acid sequences (SEQ ID NO. 1 to SEQ ID NO. 12) are provided, and the corresponding nucleotide coding sequences (SEQ ID NO. 13 to SEQ ID NO. 24) are synthesized by a gene synthesis company. The synthesis rules are based on the codon preference of the host. Due to differences in hosts, codon preferences also vary (SEQ ID NO. 13 to SEQ ID NO. 24 are the bactericidal protein encoding genes expressed in Trichoderma). An empty expression plasmid without the gene inserted is provided to the gene synthesis company, and the restriction enzyme sites for inserting the expression gene, as well as the upstream and downstream DNA sequences of the restriction sites, are informed to avoid frameshift mutations. The gene may then be directly synthesized onto the provided plasmid, constructing the final expression vector for expressing the reducing sugar oxidase, thereby eliminating steps such as PCR, restriction digestion, and ligation.

It should be noted that: the bactericidal protein amino acid sequences provided by the present disclosure (SEQ ID NO. 1 to SEQ ID NO. 12) all contain their own secretion peptide sequences at the N-terminus. They may be secreted and expressed in Trichoderma.

The bactericidal protein encoding gene sequences in the following embodiments are as shown in SEQ ID NO. 13 to SEQ ID NO. 24.

SEQ ID NO. 13:
ATGAAGACGACGTTTGCGGCTCTTATTGTGTCATTGACCTCCATTGTCTCT
GCATACCCCATCACCGGCGACGTCGTCAACTGCCGCACTGGCCCTGGCACCAGCTAC
GCCATCAAGACCAGCTACAAGAAGTCCCACGACATCAGCATCAGCTGCCAGACCAC
CGGCACCAGCGTCAACGGCAACAACATCTGGGACAAGACCGCCGACGGCTGCTACG
TCGCCGACTACTACGTCAAGACCGGCAGCAGCGGCTTCGTCACCAAGAAGTGCAGC
GCCTCTGGCGGCGGCGGCAGCTCCAGCGGCAGCTACTGCAAGACCATCAACGGCGC
TGGCGTCGACCTGATCGCCAAGTGGGAGGGCTTCGTCGCTAGCCCCAAGCCTGATCC
TATCGGCCTGCCTACCGTCGGCTACGGCCACCTCTGCCAGCAGAAGAACTGCGCCGA
GGTCAAGTACAAGTTTCCCCTGACCAAGACGACCGCCAAGGAGCTGCTCCTCGACG
ACCTGCCTAAGTACACCAAGTGCCTCGCCGACGTCCTCAACAGCAAGCCCAAGCTC
AACGCCAACCAGTGGGCCGCTCTCAGCAGCTGGGTCTTTAACGTCGGCTGCGGCAA
CGCCAAGACGAGCACCCTCGTCAAGCGCCTCAACAACGGCGAGGCCCCTAACACCG
TCGCCGCTCAGGAGCTGCCCAAGTGGCGCATGGCTGGCGGCAAGGTCATGCCCGGC
CTTGAGGCCCGACGCAAGGACGAGGTGAAGCTGTTCAAGACGGCCAGCTCCAAGGAGGCTTAC
CCCAAGTGCCAGGCC.
SEQ ID NO. 14:
ATGTCATCCAGCCTCAAGTTCCTGGGGGTCCTGCTGCCGGCGCTCGCCGTC
GCTTGCACCGGCCCTCCTGTCAACCAGAACGGCCTCAACCTCATCAAGTCCTTCGAG
AGCTTCCAGCCCAGCGTCTACGACGACGGCTTCGGCAACCCCACCATCGGCTACGGC
CACCTCTGCGGCGACGCTACTTGCAGCGAGGTCACCTACCCCAAGCCCCTGTCCGAG
GCCGACGCTTCTCGCCTCCTGGCTGACGACCTCGTCTCCTACCAGGACGCCCTCACC
AACGCCCTGGCCGATCCTGTCACCCTCAACGACAACCAGTACGCCGCCCTCGTCAGC
TGGACCTTCAACATCGGCAACGGCAACATGCAGAAGAGCGACCTGGTCGCCCGCAT
GAACAAGGGCGAGAACGTCGCCACCGTCGCCCACGATGAGCTGCCTCAGTGGAACA
AGGCCAACGGCCAGGTCGTCAACGGCCTGACCCGCCGACGAAAGGCCGAGCTCGAC
CTGTTCGACGCCCCCGCTATCTACGGCGCCCTGCCTGTCCCTTGC.
SEQ ID NO. 15:
ATGCCTTCGAACACCATTAAGTTCATCCTCGCTGCGTTGCCCGCCCTGGCG
GCAGCGGCTTGCAGCGGACCCGACGTCAACGAGGCCACCATCAAGCTCATGAAGGG
TTATGAGAGCTGGGAGGCCGACGTTTACGACGACGGCTACGGGAACCCGACAGTGG
GATATGGCCACCTCTGCGATGACTGGTCCTGTTCGGACGTATCTTACGACATTCCGC
TCTCGGAGTCAGATGGCGAGAAGCTGTTTGCGGAAGATATCGTGGCATACCAAAAC
GGCGTCGTGGCTGCTCTAAGCGACGATGTGACGCTGAACGATAATCAGTACGGCGC
CTTAGTCAGTTGGTGCTTCAACGTCGGCACGGGGGCCGTTGCGGAATCTACACTTGC
CAAGAGGCTCAACAACGGCGAAGATCCGGACACCGTCGCCGAGGAGGAGCTCCCCA
AGTGGGTCTACGCCAATGGTGCCCCATCCGAAGGCTTGAAGAATCGGCGCGCAGCA
GAGCTGAAACTGTTTACTACTAGCTCCGACACGGAGGCCCTGCCTGTCGACTGC.
SEQ ID NO. 16:
ATGCAGCGCACCGTCATTGCCGCCCTTGCTGCTCTGGCCTCCGTGGCGTCT
GCTCAGTGCAACATCCCGGATGTGAACACTGCAACGATCAACCTGATCACCGAGTTT
GAGGGCTTCCGTGCTAATATCTACACGGATCCGACGGGCAACCCGACAGTCGGCAT
TGGCCACCTCTGCCGCGACAGCCGGTGCTCTGACGTCTCGTATCCCATACCTCTCTCCCAAG
CCAACGGCAAGAAGCTGCTTGCCGACGACCTGGGCACATACGAGGAATGCCT
GACCATGATGACCAACAGCCGAGTTGTTCTGAATCTCAACGAGTTTGGGGCCCTCGT
GAGCTGGGCGTTCAACATGGGCTGCGGTGCGGCCAAAGGCTCCACGCTCATCAAGC
GGTTGAATGCAGGAGAGAAGCCCCAGACGGTGCTCCCTCAAGAACTACCCAAGTGG
GTCAATGGGAACGGACAGGTCCTGCCAGGTCTCGTAAGAAGGCGCAACGCGGAAGT
CGAGTTGGCCCGAACCGCGACTTCAAGTCCAGCACTGCCCGCCTGTTCG.
SEQ ID NO. 17:
ATGAAGTTCTTGACTCCGCTTGCTGTGCAGCTCGCCGCCCTGGCGTCCATG
GCCTCGGCGACAGTTCCCGGCTTCGACATTAGCCATTACCAGGCAACCGTCGACTTC
GCCAAGGCGTACGCCGACGGCGCCCGCTTCGTCATCATCAAGGCCACAGAAGGCAC
CACCTACACCGACCCCAGCTTTAGTGACCACTATACGAAAGCTACCAACGCCGGTTT
CATCCGAGGAGGCTACCACTTTGCCCAGCCCGCATCTTCTAGCGGGGCTGCACAGGC
CAACTACTTCCTAAAGCACGGCGGTGGCTGGTCCGCCGATGGCATAACCCTGCCGG
GCATGCTCGACCTGGAGTATGCACCTTCTGGAGATTCATGCTACGGCCTCAGTGCAT
CAGCCATGGTCTCCTGGATCAACGACTTTGTCAACACGTACCATGCCGCGACGACGC
AGTACCCTCTCATCTACACATCGACGAGCTGGTGGCAATTGTGCACTGGCAACAACG
GGTCCTTTGGCAGCAAGAGCCCACTTGTTATTGCCAGGTATGCTTCGAGCGTGGGAG
CTCTGCCCAATGGATGGTCGGTATATACCATCTGGCAGAACTCGGATGCGTCCCCGT
GGGGCGGCGACAACGATATCTTCAATGGGAATCTCGCTCAACTGCAGAAGATTGCG
CGGGGTTCA.
SEQ ID NO. 18:
ATGAAGTTCTCGATAGCTGCCATTGCCTTGTTCACCCTGAGCGCCGTGGCG
CTGCCGGCAACTGTTGTTCGGAGGGGCGCCTCTGCCGCCACCGTCAACCTCATCAGC
GAGGTCGAGGACTTCCGCGCCGACTTCTACGACATGATGGGCCACAAGACCATCGG
CTACGGCCACGACTGCGTCGCCAAGAAGGACTGCGACAGCATCAACGCCCCTATCA
GCAACGCCCAGGGCGACGAGATCCTCCACAAGGACCTCGTCGGCTTCGAGCAGTGC
GTCTGCGCCCTGCCTAACGCCAACGCTCTCAACGCCAACCAGTACGGCGCCCTCGTC
AGCTACGCCTTCAACACCGGCTGCGGCGGCCTCCAGCAGGCCTGGACCGCCGCCAT
GACCAGCAAGAACTTCGACTCCATCTGCGCCGATTTTCCCCACACCAACACGCTCAA
CGGCGTCCTCGACAACCGCCGCAAGAAGGAGGCCGCTCTCTGCAGCACGCCCACCA
CGCAGATGAGCGGCTGC.
SEQ ID NO. 19:
ATGAAGTTCAGCATAGCTGCCATCGCCCTCTTCACCCTCTCAGCCGTCGCC
TTGCCTGCCACCGTGGTTCGACGGGGCGCCTCTGCCGCCACCGTCAGCCTCATCGGC
GAGGTCGAGGGCTTCCGCGCCGACTTCTACGACATGATGGGCCACAAGACCATCGG
CTACGGCCACGACTGCGTCGCCAAGCAGGACTGCGACAGCATCAAGGCCCCTATCA
GCAACGCCCAGGGCGACGAGATCCTCCAGAAGGACCTGGCCGGCTTCGAGCAGTGC
GTCTGCGCCCTGCCTAACGCCAAGGCTCTCAACGCCAACCAGTACGGCGCCCTCGTC
AGCTACGCCTTCAACACCGGCTGCGGCGGCCTCCAGCAGGCCTGGACCGCCGCCAT
GACCAGCAAGAACTTCGACTCCATCTGCGCCGACCTGCCTCACACCAACACGCTCAA
CGGCGTCCTCGACAACCGCCGCAAGAAGGAGGCCGCTCTCTGCAGCACGCCCACCACGCAGA
TGTGCGGCTGC.
SEQ ID NO. 20:
ATGAAGACTACCTTTGCCGCTCTTATTGTTTCGTTGACGTCCATCGTGTCA
GCATATCCCATCACGGGAGATGTCGTCAACTGCCGCACGGGACCCGGCACATCGTA
CGCCATTAAGACCAGCTACAAGAAGAGCCACGACATCAGCATATCCTGCCAAACCA
CGGGGACCAGCGTCAATGGCAACAACATCTGGGACAAAACTGCTGATGGCTGCTAC
GTGGCCGACTACTACGTCAAGACGGGGTCCTCGGGCTTCGTCACCAAGAAGTGCTC
GGCGTCTGGCGGCGGGGGTGGTGGATCCTCTTCCGGGAGCTACTGCAAGACAATCA
ACGGCGCCGGAGTGGATCTCATCGCGAAGTGGGAGGGCTTTGTTGCCTCACCTAAG
CCCGACCCGATTGGCTTGCCGACAGTTGGCTACGGCCATCTATGCCAGCAGAAGAA
CTGTGCTGAGGTAAAGTACAAGTTCCCTCTGACCAAAACGACCGCCAAGGAACTGC
TGCTCGACGACCTGCCGAAGTATACGAAATGCCTGGCCGAGGTGTTGAATAGCAAA
CCAAAGCTGAACGCAAACCAGTGGGCTGCCCTGTCGAGCTGGGTCTTCAACGTGGG
ATGCGGCAATGCGAAGACCTCTACACTTGTCAAGCGACTCAACAATGGTGAGGCTC
CCAACACCGTGGCAGCGCAGGAGCTCCCAAAATGGCGCATGGCCGGCGGCAAGGTC
ATGCCCGGCCTCGAGGCGAGGCGGAAGGACGAAGTCAAGCTCTTCAAGACGGCCAG
TTCAAAGGAAGCATATCCGAAATGTCAAGCC.
SEQ ID NO. 21:
ATGAAGACGGCCTTTGCTGCTCTTGTTTTTAGCCTGGCAAGCATTGTCTCC
GCCTACCCCATCACAGGCGATGTCGTGAATTGTCGAACTGGTCCGGGAACCTCCTAC
GCTATCAAGAAAAGCTACAAGAAGAACCAAGATATTAGCATCTCGTGCCAGACCGC
AGGGACGTCCGTCAACGGCAACTCCATCTGGGACAAGACTGCGGACGGCTGCTACG
TGGCCGACTACTACGTCAAGACCGGTAGTTCTGGATATGTCACCAAGAAGTGCAGC
GCCTCGTCCGGAGGTGGCTCATCTAGTTCATCGTACTGCAAGACCATCAACACTGCT
GGAGTCGACTTGATAGCCAAATGGGAGGGCTTCGTAGCCAGCCCCAAGCCCGATCC
TATTGGGCTGCCAACAGTGGGCTATGGGCACTTGTGCCAGCAGAAGAACTGCAGAG
AAGTGAAATACAAGTTCCCGCTGACTAAAACGACGGCGAAGGAGCTGCTGCTCGAC
GACCTTCCAAAGTACACGAAGTGCCTCGCCGATTATCTAAACGACAAGCCGAAGCT
GAATGCCAACCAGTGGGCGGCGCTCACATCCTGGGTGTTCAACGTCGGCTGTGGCA
ATGCCAAAACCTCTACCCTCGTCAAGCGGCTCAACAACGGCGAGGCGGCAAACACG
GTTGCCGCCGAGGAGCTCCCCAAGTGGCGCATGGCCGGCGGCAAGGTCCTGCCTGG
CCTGGAAGCGAGGCGCAAGGACGAGGTTAAGCTCTTCAAGACGGCATCGTCGAAGC
AGGCTTATCCCAAGTGCCAA.
SEQ ID NO. 22:
ATGAAGACGGCCTTTGCCGCGCTGGTGTTTTCCCTTGCCTCGATTGTCTCG
GCGTATCCCATCACCGGCGACGTCGTCAACTGCAGGACGGGGCCGGGGACAAGCTA
TGCCATCAAGAAATCGTACAAGAAGAACCAAGACATATCCATCAGCTGCCAGACGG
CTGGCACGTCTGTCAATGGCAACAGCATCTGGGACAAGACTGCGGATGGCTGCTAC
GTCGCTGACTACTATGTGAAAACTGGTAGTAGTGGATACGTGACGAAGAAGTGCAC
CGCATCTTCAGGTGGAGGAAGCTCGTCATCTTCCTACTGCAAGACCATCAACACGGC
GGGAGTCGACCTCATTGCCAAGTGGGAGGGCTTCGTGGCCAGCCCGAAGCCCGATC
CTATTGGGCTCCCCACGGTTGGCTACGGCCACCTCTGCCAGCAGAAGAATTGCCGCG
AAGTTAAGTACAAGTTCCCTCTGACCAAGACCACAGCAAAGGAGCTCCTCCTGGAC
GATTTGCCGAAATACACCAAATGCTTGGCCGATTATCTCAACGACAAGCCAAAGCT
GAACGCCAACCAGTGGGCTGCTCTTACTAGCTGGGTCTTCAACGTTGGCTGTGGCAA
TGCCAAGACGTCGACCCTCGTCAAGCGACTTAACAACGGCGAGGCAGCAAACACCG
TGGCGGCCGAGGAGCTGCCAAAATGGCGCATGGCCGGCGGCAAGGTCCTGCCCGGT
CTGGAAGCCAGACGGAAGGACGAGGTAAAGCTCTTCAAGACAGCTTCCTCCAAGCA
GGCGTACCCTAAGTGTCAA.
SEQ ID NO. 23:
ATGAGCTCATTCAAGCTGCTTGCTGCATTGCTCCCAGCACTGGCTGTGGCC
TGCACTGGACCTCCCGTCAACCAAAACGGTCTGAACCTCATCAAGTCGTTTGAGTCC
TTCCAGCCGTCTGTATACGACGACGGCTTCGGCAATCCCACCATTGGCTATGGCCAT
CTTTGCGGCGACGCCACCTGCTCCGAAGTGACGTATCCCAAGCCCTTGTCGGAAGCG
GATGCATCCGCACTACTGGCCGGGGATCTGGTCAGCTACCAGGATGCTCTCACCAAC
GCGCTCGCCGACCCCGTCACGCTGAACGACAACCAGTACGCCGCGCTTGTGAGTTG
GACGTACAACGTGGGTAACGGAAATATGCAGAAGAGCGACCTCGTCGCGCGCATGA
ACAAGGGCGAGGACGTCGCCGCCGTTGCGCACGACGAGCTCCCTCAATGGAACAAA
GCTAACGGCCAGGTCGTCAATGGGCTCACAAGGCGACGGAATGCCGAGCTGGGACT
GTTTGATGCTCCAGCCATCTACGGCGCCTTGCCGGTTCCGTGT.
SEQ ID NO. 24:
ATGAAAGCCATCTCCCTCCTTGCACTACCATCTCTCGCTTATGCTGCTGTC
CAGGGCTTCGACATTTCCAATTGGCAACCGACAGTCGACTACGACGGCGCGTATGC
GTCCGGTGCCCGATTTGCCATGATCAAGGCGACCGAAGGCACCACCTACATCGACC
CCCTCTTCAACACGCACTACCCTGGCGCTACCAGCGCCGGCCTCATACGGGGAGGTT
ACCACTTCGCCCATCCCGATTCTTCGTCGGGGTCAGCACAGGCCTCCTACTTCTTGGCGCA
CGGCGGAGGCTGGTCCGCAGATGGGCTGACGCTGCCCGGCATGCTCGACCTGG
AAGCCGGCTGCTATGGCCTGAGCCCGTCGGCCATGACGAGCTGGATCCGCGACTTC
GGAGAGACGTACAAGGACGCCACCGGGAGATATCCCATGATTTACACGACTACATC
ATGGTGGACCGAGTGCACCGCCAACAACGCCGATTTTGGCGACTATCCACTCGTGTT
GGCAAGGTGGGCCTCTTCCCCGGGCACACTGCCTGCGTCGTGGGATTACTACAGCTT
CTGGCAGAATAGCGACCAGTACGCCTTTGGTGGAGATAGCCAAGTCTGGAACGGCA
GCGAGGAGCGTCTTCGCGTTTTTGCTTCGGGG.

Similar to lysozyme extracted from commercial chicken egg white, the bactericidal protein hydrolyzes the cell wall of the indicator bacterium, causing the rupture and death of the indicator bacterium. The process manifests as a gradual decrease in the absorbance of the indicator bacterium suspension, and the suspension slowly becomes clear. Therefore, in the following embodiments, the national standard GB1886.257-2016 assay method is used to determine the bactericidal activity of the bactericidal protein. The bactericidal activity is simply defined as follows: one unit of bactericidal activity is defined as the volume or mass of the bactericidal protein solution required to cause a decrease in absorbance of 0.001 per minute at 450 nanometers in a Micrococcus luteus suspension at 25 degrees Celsius and pH 6.2. The level of bactericidal activity may also directly reflect the expression level of the bactericidal protein.

### Embodiment 1 Construction of Bactericidal Protein Expression Vectors

The present disclosure utilizes a previously constructed Trichoderma reesei expression plasmid CBHIT1.0 (described in the patent document 202211596367.5 Construction and Application of Xylanase and Corresponding Secretion Expression Strain) to construct recombinant Trichoderma strains, enabling the secretion and expression of bactericidal proteins in the recombinant Trichoderma strains.

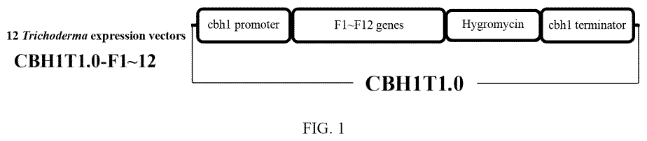

The present disclosure commissions Shanghai Generay Biotech Co., Ltd. to synthesize the 12 bactericidal protein encoding gene sequences directly into the XbaI restriction site (TCTAGA) of the CBH1T1.0 plasmid, thereby constructing the corresponding 12 recombinant expression vectors, named in order as CBHIT1.0-F1˜12 (a schematic diagram of the recombinant expression vector structure is shown in FIG. 1).

The upstream and downstream sequences near the XbaI restriction site (the target gene insertion site) on the CBHIT1.0 plasmid are shown in SEQ ID NO. 25. The gene insertion site TCTAGA is underlined, and the insertion position is indicated by “/”. The ATG at the front of the bactericidal protein encoding gene sequence is the start codon of the encoding gene, and the TAG in the latter part of the XbaI restriction site on the CBHIT1.0 plasmid is the stop codon.

SEQ ID NO. 25:
TTCAGGGGCCACTGCATGGTTTCGAATAGAAAGAGAAGCTTAGCCAAG
AACAATAGCCGATAAAGATAGCCTCATTAAACGGAATGAGCTAGTAGG
CAAAGTCAGCGAATGTGTATATATAAAGGTTCGAGGTCCGTGCCTCCC
TCATGCTCTCCCCATCTACTCATCAACTCAGATCCTCCAGGAGACTTG
TACACCATCTTTTGAGGCACAGAAACCCAATAGTCAACCGCGGACTGC
GCATC/TAGATAACTTCGTATAATGTATGCTATACGAAGTTATACTAG
AGAAGTTCCTATACTTTTTAGAGAATAGGAACTTCGGAATAGGAACTT
CACTAGAGATATCAAGCAACTACGTAAAACTCCATGAGATTGCAGATG
CGGCCCACTGGAATACAACATCCTCCGCAAGTCCGA.

### Embodiment 2 Construction of Recombinant Trichoderma Strains Expressing Bactericidal Proteins

The 12 recombinant expression vectors constructed in Embodiment 1 are used to transform Trichoderma strains QM6a (ATCC 13631), QM9414 (ATCC 26921), RUT-C30 (ATCC 56765), RL-P37 (NRRL 15709), NG14 (ATCC 56767), and PC-3-7 (ATCC 66589) respectively via the Agrobacterium tumefaciens-mediated method (Covert et al. Agrobacterium Tumefaciens-mediated Transformation of Fusarium Circinatum. Mycol. Res. 105 (3): 259-264), thereby obtaining recombinant Trichoderma strains.

In this embodiment, the Trichoderma strain RUT-C30 (ATCC 56765) is used as the host strain to illustrate in detail the process of constructing recombinant Trichoderma strains—RUT-C30-CBHIT1.0-F1˜12. When other Trichoderma strains are used as host strains, the construction process is completely identical.

The process is as follows.

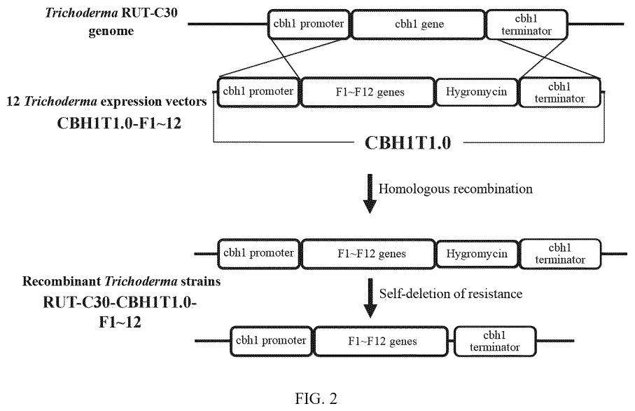

The recombinant expression vectors CBHIT1.0-F1˜12 are transformed into the Trichoderma strain RUT-C30 using the Agrobacterium tumefaciens-mediated method, with homologous recombination replacing the endogenous cellulase gene cbh1, thereby successfully constructing 12 recombinant Trichoderma strains expressing bactericidal proteins respectively (a schematic diagram of the construction process is shown in FIG. 2). The hygromycin resistance markers are eliminated from the recombinant Trichoderma strains according to the literature protocol (Zhang et al. Light-inducible Genetic Engineering and Control of Non-homologous End-joining in Industrial Eukaryotic Microorganisms: LML 3.0 and OFN 1.0. Scientific Reports. 2016, 6:20761).

### Embodiment 3 Production of Bactericidal Proteins

The recombinant Trichoderma strains are fermented and cultured in Trichoderma fermentation medium to induce the secretion and expression of bactericidal proteins. The fermenter cultivation method refers to the literature Chen et al. Engineering of Trichoderma Reesei for Enhanced Degradation of Lignocellulosic Biomass by Truncation of the Cellulase Activator ACE3. Biotechnol Biofuels. 2020, 13:62. After 8 days of fermentation, the fermentation broth is collected, centrifuged at 12,500 revolutions per minute for 10 minutes, and the supernatant is taken, which is the crude extract of bactericidal proteins. The crude extract is spray-dried, and then a universal protective agent (starch or cyclodextrin) is added at a weight ratio of 1:1 to produce solid fungal bactericidal protein powder. The national standard GB1886.257-2016 is used to determine the activity of the crude extract and the fungal bactericidal protein powder, respectively. As shown in Table 1, the activity of the fungal bactericidal protein powder reaches 100-400 units per milligram

TABLE 1
Bactericidal activity of bactericidal proteins expressed by recombinant Trichoderma
strains (Staphylococcus aureus ^{a} is the abbreviation for Staphylococcus aureus)

| | Bactericidal activity | | | | |
|---|---|---|---|---|---|
| | Crude extract | Fungal bactericidal | Inhibition zone diameter | | |
| Recombinant | (units per | protein powder | Candida | Staphylococcus | Escherichia |
| Trichoderma strain | milliliter) | (units per milligram) | albicans | aureus ^{a} | coli |
| RUT-C30-CBH1TI.0-F1 | 623 | 238 | 1.4 ± 0.2 | 1.8 ± 0.3 | 1.2 ± 0.2 |
| RUT-C30-CBH1TI.0-F2 | 845 | 287 | 1.0 ± 0.2 | 1.4 ± 0.2 | 1.6 ± 0.2 |
| RUT-C30-CBH1TI.0-F3 | 867 | 221 | None | 1.2 ± 0.2 | 1.7 ± 0.1 |
| RUT-C30-CBH1TI.0-F4 | 976 | 246 | None | 1.8 ± 0.2 | 1.8 ± 0.1 |
| RUT-C30-CBH1TI.0-F5 | 1352 | 356 | 1.4 ± 0.2 | 1.5 ± 0.2 | 1.3 ± 0.1 |
| QM6a-CBH1TI.0-F5 | 245 | — | — | — | — |
| QM9414-CBH1TI.0-F5 | 524 | — | — | — | — |
| RL-P37-CBH1TI.0-F5 | 1086 | — | — | — | — |
| NG14-CBH1TI.0-F5 | 825 | — | — | — | — |
| PC-3-7-CBH1TI.0-F5 | 1305 | — | — | — | — |
| RUT-C30-CBH1TI.0-F6 | 768 | 305 | 1.2 ± 0.2 | 1.8 ± 0.1 | 1.5 ± 0.1 |
| RUT-C30-CBH1TI.0-F7 | 1068 | 316 | None | 1.5 ± 0.2 | 1.3 ± 0.1 |
| RUT-C30-CBH1TI.0-F8 | 657 | 185 | 1.7 ± 0.3 | 1.5 ± 0.2 | 1.6 ± 0.1 |
| RUT-C30-CBH1TI.0-F9 | 645 | 208 | 1.9 ± 0.1 | 1.2 ± 0.2 | None |
| RUT-C30-CBH1TI.0-F10 | 357 | 158 | None | None | 1.1 ± 0.2 |
| RUT-C30-CBH1TI.0-F11 | 586 | 295 | None | None | 1.8 ± 0.1 |
| RUT-C30-CBH1TI.0-F12 | 784 | 287 | 1.1 ± 0.2 | 1.1 ± 0.2 | 1.5 ± 0.1 |

Table 1 shows that when the Trichoderma strain RUT-C30 (ATCC 56765) is used as the host strain, the activity of the produced bactericidal protein is higher.

### Embodiment 4 Application of Bactericidal Proteins

The bactericidal protein powder prepared in Embodiment 3 is used for the inhibition zone assay, with commercial chicken egg white-extracted lysozyme powder (sourced from Ningxia Xiasheng Industrial Group Co., Ltd., activity ˜3000 units per milligram) as the control. The inhibition effect of the bactericidal protein on common pathogenic bacteria is evaluated through the inhibition zone assay method.

The experimental method is as follows.

Taking the bactericidal protein powder F5 produced by RUT-C30-CBHIT1.0-F5 as an example, an appropriate amount of F5 and commercial lysozyme powder are dissolved separately in ultrapure water to prepare a test solution and a control solution with a concentration of 40 units per microliter, followed by sterilization through filtration. Three indicator bacteria are selected: Candida albicans, Staphylococcus aureus, and Escherichia coli. The indicator bacteria are streaked on LB plates and cultured at 37 degrees Celsius for about 12 hours. Single colonies are picked from the plate into an LB test tube and cultured in a shaker at 37 degrees Celsius for about 12 hours to prepare indicator bacterium seed cultures, which are stored in a refrigerator for later use.

After the LB medium is sterilized, the LB medium is cooled to a temperature slightly above body temperature. Then, 100 microliters of each of the above indicator bacterium seed cultures is pipetted into the un-solidified LB medium, mixed well, and quickly poured into plates, with 15 milliliters of medium per plate, and allowed to solidify.

Filter paper discs with a diameter of 6 millimeters are cut, sterilized at high temperature, and dried aseptically. Each filter paper disc is made to absorb 10 microliters of the bactericidal protein test solution or the commercial lysozyme control solution. Sterilized tweezers are used to pick up the filter paper discs, which are then placed onto the surface of the plates. The plates are inverted, labeled, and placed in a 37 degrees Celsius incubator for cultivation. Inhibition zones may be observed after about 12 to 24 hours.

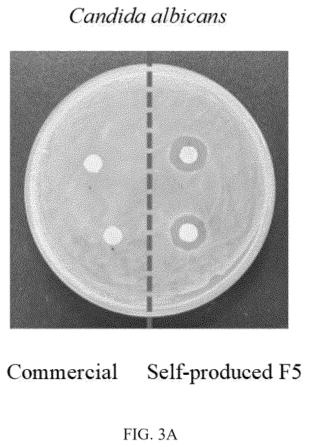

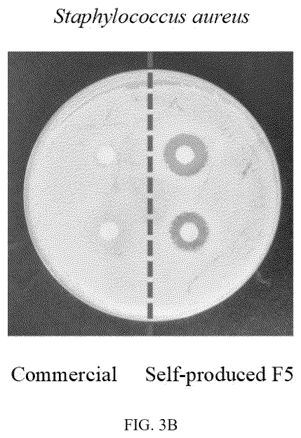

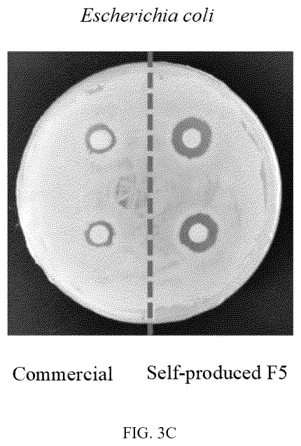

The inhibition zone experimental results are shown in FIG. 3A, FIG. 3B and FIG. 3C. It may be seen that the bactericidal protein may significantly kill or inhibit the growth of Gram-positive bacteria (Staphylococcus aureus is resistant to chicken egg white lysozyme) and yeasts (Candida albicans), producing significant inhibition zones, whereas chicken egg white lysozyme may not inhibit these two strains. The bactericidal protein may significantly kill or inhibit Gram-negative bacteria (Escherichia coli), and its effect exceeds that of chicken egg white lysozyme, producing larger inhibition zones. The sizes of inhibition zones produced by other bactericidal protein powders are shown in Table 1.

In summary, after genetic recombination modification of Trichoderma, the present disclosure successfully constructs recombinant Trichoderma strains that secrete and express bactericidal proteins. The bactericidal proteins produced by these recombinant Trichoderma strains may significantly kill or inhibit Gram-positive bacteria, Gram-negative bacteria, and yeasts, providing a new bactericidal protein preparation and a preparation method thereof for industries including daily chemicals, food, and feed.

The embodiments described above are only illustrative of optional implementations of the present disclosure and are not intended to limit the scope of the disclosure. Without departing from the design spirit of the present disclosure, various modifications and improvements to the technical schemes of the present disclosure made by those skilled in the art shall fall within the scope of protection defined by the claims of the present disclosure.

What is claimed is:

1. A bactericidal protein, wherein an amino acid sequence of the bactericidal protein is any one of SEQ ID NO. 1 to SEQ ID NO. 12, or a sequence having identical function after 1 to 30 amino acid substitutions and/or deletions and/or additions from any one of the SEQ ID NO. 1 to the SEQ ID NO. 12, or a sequence having identical function after adding 1 to 20 amino acids to C-terminus and/or N-terminus of any one of the SEQ ID NO. 1 to the SEQ ID NO. 12.

2. A recombinant microorganism, wherein the recombinant microorganism comprises a recombinant expression vector carrying a gene encoding the bactericidal protein according to claim 1; and a nucleotide sequence of the gene is any one of SEQ ID NO. 13 to SEQ ID NO. 24, or a sequence having more than 85% homology with any one of the SEQ ID NO. 13 to the SEQ ID NO. 24.

3. A method for producing a bactericidal protein, comprising fermenting and culturing the recombinant microorganism according to claim 2, collecting a fermentation broth, and obtaining the bactericidal protein.
